(19) United States
(12) Patent Application Publication
(10) Pub. No.: US 2026/0345456 A1
(43) Pub. Date: Nov. 5, 2026

(54) ALCOHOLIC EXTRACT OF WHITE LILY FLOWERS LILIUM CANDIDUM L, METHOD FOR OBTAINING SAME, AND COSMETIC COMPOSITION CONTAINING SAME

(71) Applicant: CHANEL PARFUMS BEAUTE, NEUILLY SUR SEINE (FR)

(72) Inventors: David LEGANGNEUX, PANTIN (FR); Maéva GILLET, PANTIN (FR)

(73) Assignee: CHANEL PARFUMS BEAUTE, NEUILLY SUR SEINE (FR)

(21) Appl. No.: 19/117,694

(22) PCT Filed: Oct. 5, 2023
(86) PCT No.: PCT/EP2023/077617

§ 371 Date: Apr. 2, 2025

(30) Foreign Application Priority Data
Oct. 5, 2022 (EP) 22306491.6

(51) Int. Cl. A61K 8/60; A61K 8/06; A61K 8/44; A61Q 19/08

(52) U.S. Cl. CPC A61K 8/602; A61K 8/062; A61K 8/442; A61Q 19/08; A61K 2800/591

(57) ABSTRACT

An alcoholic extract of white lily flowers Lilium candidum L., and a method for extracting white lily flowers Lilium candidum L in the presence of an alcoholic solvent. Also, a cosmetic composition containing the alcoholic extract of white lily flowers Lilium candidum L., as well as different cosmetic uses of the extract of white lily flowers Lilium candidum L.

### FIELD

The invention relates to an extract of flowers of white lily, Lilium candidum L., to a method for obtaining same, to a cosmetic composition containing same, and also to various cosmetic uses.

### BACKGROUND

The skin consists mainly of three layers, namely, starting from the most superficial, the epidermis, the dermis and the hypodermis.

The epidermis contributes greatly to protecting the skin and to maintaining the correct functioning thereof.

Aging and photoaging of the skin and the detrimental changes associated therewith can manifest themselves in a number of ways, including:
loss of firmness and elasticity due to tissue loss in the epidermis and/or dermis;
loss of radiance due to reduced microcirculation and to a slowing of cell renewal in the epidermis;
the appearance of pigment spots; and/or
dryness of the skin resulting from a decrease in the barrier function of the stratum corneum and a slowing of epidermal renewal.

There is, therefore, a need to provide a polyfunctional active agent capable of acting on a set of causes of detrimental skin changes due to aging and/or to a modification of the physiological mechanisms related to aging.

### SUMMARY

In point of fact, the applicant has now found that an alcoholic extract of flowers of white lily, Lilium candidum L., obtained by a particular method exhibits, by means of stimulation or inhibition of physiological mechanisms, advantageous activities with regard to skin aging, in particular via protective properties against oxidative stress giving it an antioxidant potential. Indeed, as demonstrated in examples, the alcoholic extract of flowers of white lily, Lilium candidum L., according to the invention has advantageous cosmetic properties: it allows activation of the biological pathway for “NRF2-mediated oxidative stress response”. In addition, aging of the dermis is characterized in particular by the progressive differentiation of papillary fibroblasts into reticular fibroblasts leading to stiffening of the dermis. However, the alcoholic extract of flowers of white lily, Lilium candidum L., according to the invention allows an increase in the expression of the CCRL1, NTN and PDPN papillary markers and also a decrease in the expression of the CDH2, CNN and MGP reticular markers in reticular fibroblasts.

The extract according to the invention also has an anti-aging effect on the skin, since it makes it possible to improve the morphology of the epidermis, in particular by promoting its thickening in models of aged skin reconstructed with reticular fibroblasts.

According to a first aspect, the invention therefore relates to an alcoholic extract of flowers of white lily, Lilium candidum L., comprising at least lilidine glycoside and hopantenic acid.

According to a second aspect, the invention also relates to a method for extracting flowers of white lily, Lilium candidum L., comprising at least the following steps:
a) milling the flowers of white lily, Lilium candidum L.;
b) at least one, preferably at least two, extractions of said milled flowers in the presence of an alcoholic solvent, each extraction being followed by a filtration step;
c) mixing the liquid filtrates from the extractions of step b) and resting of the resulting mixture for at least 6 h, preferably at least 10 h;
d) filtering the mixture obtained in c);
e) removing the monoalcoholic solvent from the mixture obtained in d) and final diluting in another alcoholic solvent.

The invention also relates to an alcoholic extract of flowers of white lily, Lilium candidum L., which can be obtained by such a method.

In addition, the invention also relates to a cosmetic composition comprising, in a cosmetically acceptable carrier, an alcoholic extract of flowers of white lily, Lilium candidum L., according to the invention. The term “cosmetically acceptable carrier” is understood to mean a medium compatible with the skin, mucous membranes and superficial body growths. Preferably, the cosmetic composition according to the invention is suitable for topical application.

### BRIEF DESCRIPTION OF THE FIGURES

FIG. 1 illustrates the relative expression (vs untreated condition) of papillary markers (CCRL1, NTN, PDPN) and reticular markers (CDH2, CNN, MGP) in reticular fibroblasts (ret) treated with the lily extract at 0.25% for 96 h (n=3 donors D1 to D3).

FIG. 2 illustrates the overall morphology of skins reconstructed with papillary fibroblasts (left) or reticular fibroblasts (center and right), which are untreated (control, left and center) or treated with the lily extract at 0.25% (right) on n=3 donors (D1 to D3). The white square brackets illustrate the differences in thickness of living epidermis observed.

FIG. 3 indicates the percentage of cells expressing the Ki67 proliferation marker in reconstructed skins comprising untreated papillary fibroblasts (control), and papillary fibroblasts treated with the control solvent (propanediol) or treated with the lily extract at 0.25% (n=3 donors D1 to D3).

### DETAILED DESCRIPTION

The starting material used consists of fresh or dried flowers of white lily, Lilium candidum L.

Lilium candidum L. is a herbaceous plant of the family Liliaceae, called the white lily or Madonna lily.

The Madonna lily is a bulbous perennial with fleshy scales, of the family Liliaceae, which can be up to 1.8 m high.

The scaly bulb produces basal leaves, forming a rosette.

The leaves, carried by sturdy stems, are shiny, pointed, alternating and decreasing in size.

The spectacular flowering offers 5 to 10 trumpet-shaped, immaculate white flowers, 15 cm long, composed of 6 large tepals, a long pistil and 6 long stamens. Flowering takes place in June.

The Madonna lily is grown in France.

The dry extract of Lilium candidum L. which is the subject of the present invention is composed of several families of primary and secondary metabolites of interest.

The alcoholic extract of flowers of white lily, Lilium candidum L. which is the subject of the present invention comprises in particular at least lilidine glycoside and hopantenic acid.

In particular, the alcoholic extract of Lilium candidum L. of the invention comprises:
40 to 70% by weight of saccharides, preferably 50 to 60% by weight,
1 to 5% by weight of compounds derived from di-O- and tri-O-glycosylated kaempferol, preferably 2.5 to 3.2% by weight,
8 to 15% by weight of lilidine glycoside, preferably 10 to 13% by weight, and
1 to 5% by weight of hopantenic acid, preferably 2 to 3% by weight,
the percentages being expressed by weight, based on the total weight of the extract.

Di-O- and tri-O-glycosylated kaempferol derivatives are compounds of following structure I (which corresponds to kaempferol tri-O-glycopyranoside) or II (which corresponds to kaempferol di-O-glycopyranoside):

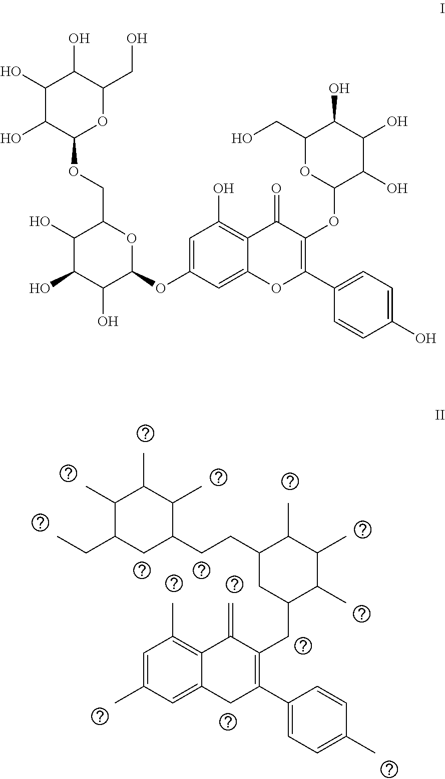

Lilidine glycoside is a compound corresponding to the following formula III:

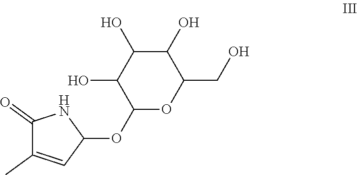 [SMILES: CC1=CC(OC2OC(CO)C(O)C(O)C2O)NC1=O]

Hopantenic acid, for its part, corresponds to the following structure IV:

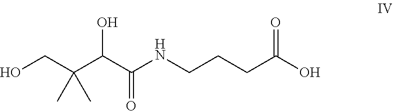 [SMILES: CC(C)(CO)C(O)C(=O)NCCCC(=O)O]

The saccharides present in the extract of lily according to the invention preferably comprise glucose, fructose and/or sucrose, and in particular 20 to 25% by weight of fructose, 17 to 21% by weight of glucose and 5 to 11% by weight of sucrose.

It is to the credit of the applicant to have succeeded in preparing an extract of lily comprising in particular lilidine glycoside, hopantenic acid and preferentially derivatives of di-O- and tri-O-glycosylated kaempferol, thereby giving it its specificity.

The alcoholic extract of Lilium candidum L. of the invention can be obtained in particular by means of a method for extracting the flowers of white lily, Lilium candidum L., comprising the following steps:
a) milling the flowers of white lily, Lilium candidum L.,
b) at least one, preferably at least two, extractions of said milled flowers in the presence of an alcoholic solvent, each extraction being followed by a filtration step;
c) mixing the liquid filtrates from the extractions of step b) and resting of the resulting mixture for at least 6 h, preferably at least 10 h;
d) filtering the mixture obtained in c);
e) removing the monoalcoholic solvent from the mixture obtained in d) and final diluting in another alcoholic solvent.

Preferably, the white lily flowers used in step a) are fresh flowers or dried flowers.

They are in particular milled or reduced to pieces, for example to a fineness of less than 2 cm, by any means known to a person skilled in the art.

In step b), the milled flowers of white lily are subjected to at least one, preferably at least two, extractions with one or more alcoholic solvents, for example chosen from:
C₁-C₄ monoalcohols, such as for example methanol, ethanol or isopropanol; and
diols, such as for example propylene glycol, 1,3-propanediol or dipropylene glycol.

Preferably, the alcoholic solvent is a monoalcohol comprising from 2 to 4 carbon atoms, more preferentially ethanol.

Preferably, the alcoholic solvent is used as a mixture of at least 90% with water, preferably at least 95% with water. Preferably, the alcoholic solvent is used as a mixture between 90% and 99% with water, preferably between 95% and 99% with water.

Preferably, ethanol is used as a mixture of at least 90% with water, preferably at least 95% with water. Preferably, ethanol is used as a mixture between 90% and 99% with water, preferably between 95% and 99% with water.

In particular, ethanol is used as a 96% mixture with water.

The extraction is generally carried out by immersing and gently stirring the white lily flowers in one or more of the solvents mentioned above for a period of between 1 and 6 hours at a temperature of between 40 °C and 60 °C, preferably between 45 and 55 °C.

The extraction step b) is followed by a filtration, for example on a 50 μm screen, to remove plant residues.

According to a preferred embodiment, step b) implements at least two extractions. In this case, each extraction is followed by a filtration step.

At the end of extraction step(s) b), the liquid filtrates are recovered and mixed. The filtrate mixture is then left to stand for at least 6 h, preferably at least 10 h.

The filtrate or filtrate mixture obtained at the end of step c) is then filtered again in order to remove the insoluble substances: this is step d). Preferably, the filtration of the extract obtained in c) is carried out on a 4 μm filtration screen or membrane. A liquid filtrate is thus obtained.

Finally, the solvent present in the liquid filtrate is removed, and the remainder of the filtrate is diluted in another alcoholic solvent (step e)).

The order of the steps of removing the solvent and diluting the filtrate in another solvent is interchangeable. In particular, the solvent present in the liquid filtrate can be removed, and then the remainder of the filtrate is diluted in another alcoholic solvent. Alternatively, the liquid filtrate is first diluted in another alcoholic solvent, and the solvents are then removed. Again alternatively, the dilution of the filtrate and the removal of the solvents can be simultaneous. The alcoholic solvent used in step e) is known as “other alcoholic solvent” because it is different than the alcoholic solvent used in step a). Taking this limitation into account, the alcoholic solvent is typically chosen from the same group as that of step a), i.e. from C₁-C₄ monoalcohols and diols.

Preferably, the final dilution is carried out in a diol, preferably 1,3-propanediol. Preferably, the removal of the solvent in step e) is carried out by evaporation, in particular by vacuum evaporation.

Preferably, between steps d) and e), a step of decolorizing the filtrate obtained in d) is added. The decolorizing can be carried out by adsorption of the pigments present in the filtrate on active charcoal. This decolorizing step can be followed by a step of filtering the decolorized filtrate obtained, in particular to 1 μm.

Preferentially, the alcoholic extract of flowers of white lily, Lilium candidum L., according to the invention can be obtained by a method comprising the following steps:
a) milling the flowers of white lily, Lilium candidum L.;
b) at least one, preferably at least two, extractions of said milled flowers in the presence of an alcoholic solvent, each extraction being followed by a filtration step;
c) mixing the at least two liquid filtrates from the two extractions of step b) and resting of the resulting mixture for at least 6 h, preferably at least 10 h;
d) filtering the mixture obtained in c) so as to obtain a filtrate;
   decolorizing the filtrate obtained in d) by adsorption on active charcoal; then
   filtering the decolorized filtrate on a 1 μm membrane; and
e) removing ethanol from the resulting filtrate by evaporation, then final diluting in 1,3-propanediol.

Advantageously, the extract used according to the invention is a light color.

Also, the extract is in a sufficiently concentrated form to be able to be used without causing the formulation problems usually encountered at the concentrations required to obtain an activity in cosmetic or dermatological compositions in emulsion form, and without exhibiting a dark color, unlike plant extracts obtained by usual methods, when they are in concentrated form.

In particular, after evaporation of the ethanol, the extract of flowers of white lily, Lilium candidum L., comprises 15 to 25% by weight of dry extract of lily and 75 to 85% by weight of 1,3-propanediol, and preferably approximately 20% by weight of dry extract of lily and 80% by weight of 1,3-propanediol.

As a result, the extract according to the invention can be used directly for the preparation of a cosmetic composition.

According to another aspect, the invention relates to the cosmetic use of an alcoholic extract of flowers of white lily, Lilium candidum L., according to the invention, as an antioxidant and/or anti-aging agent.

According to yet another aspect, the invention relates to the cosmetic use of an alcoholic extract of flowers of white lily, Lilium candidum L., according to the invention, for preventing and/or reducing detrimental skin changes due to aging. Furthermore, said extract is in particular used as an agent for activating the NRF2-mediated oxidative stress response and/or an agent promoting epidermal thickening.

Indeed, it has been found, advantageously, that the alcoholic extract of flowers of white lily, Lilium candidum L., according to the invention possesses several activities of interest with regard to preventive or reparative physiological mechanisms linked to detrimental skin changes, due in particular to aging.

It has in particular been found, advantageously, that the alcoholic extract of flowers of white lily, Lilium candidum L., according to the invention allows an increase in the expression of the CCRL1, NTN and PDPN papillary markers and also a decrease in the expression of the CDH2, CNN and MGP reticular markers in reticular fibroblasts.

The extract according to the invention also makes it possible to improve the morphology of the epidermis, in particular by promoting its thickening in models of aged skin reconstructed with reticular fibroblasts.

According to yet another aspect, the invention also relates to a cosmetic composition comprising, in a cosmetically acceptable carrier, an alcoholic extract of flowers of white lily, Lilium candidum L., according to the invention. Preferably, said extract is present in the cosmetic or dermatological composition in a proportion of 0.001 to 10% by weight, relative to the total weight of the composition, in particular in a proportion of 0.01 to 10% by weight, preferably of 0.1 to 10% by weight. Said cosmetic composition can in particular be suitable for topical application.

Advantageously, said cosmetic composition may be in the form of a powder, an emulsion, a microemulsion, a nanoemulsion, a suspension, a solution of a lotion, a cream, a gel, which may be aqueous or aqueous-alcoholic, a foam, a serum, an aerosol solution or dispersion, or a dispersion of lipid vesicles.

In the case of an emulsion, it may be a water-in-oil or oil-in-water emulsion.

The cosmetic composition according to the invention may also comprise a solvent chosen according to the various ingredients and the administration form.

By way of examples, mention may be made of water (preferably demineralized water or floral waters), or an alcohol such as ethanol.

Said cosmetic composition may also comprise, in addition to the extract according to the invention, at least one additive customary in the field, such as, for example, at least one compound chosen from an emollient or humectant, a gelling and/or thickening agent, a surfactant, an oil, an active agent, a dye, a preservative, an antioxidant, an active agent, an organic or inorganic powder, a sunscreen and a fragrance;
one or more humectant(s), such as polyols (glycerol, diglycerol, propylene glycol, propanediol, caprylyl glycol, pentylene glycol, hexanediol), sugars, glycosaminoglycans such as hyaluronic acid and its salts and esters; and polyquaterniums such as Lipidure PMB.

Said humectant will be present in the composition in a content of about from 0.1% to 30%, preferably 0.005% to 10%, by total weight of the composition;
one or more emollient(s) which may be chosen, for example, from esters such as jojoba esters, fatty acid esters of a fatty alcohol (octyldodecyl myristate, triethylhexanoin, dicaprylyl carbonate, isostearyl isostearate, caprylic/capric triglyceride), butters such as shea butters (Butyrospernum parkii butter extract, shea butter ethyl esters, sold under the names Lipex Sheasoft, Lipex Shea-U, Lipex Shea, Lipex Shealight, Lipex Shea Tris) or moringa butters (moringa oil/hydrogenated moringa oil esters), waxes (Acacia decurrens flower wax & Helianthus annuus cera seed wax, C10-18 triglycerides), plant oils, phytosqualane, alkanes (undecane, tridecane).

Said emollient will be present in the composition in a content of about from 0.1% to 30%, preferably 0.5% to 10%, by total weight of the composition;
one or more gelling and/or thickening agent(s) for the aqueous phase, chosen for example from cellulose derivatives, gums of plant origin (guar, carob, alginates, carrageenans, pectins), of microbial origin (xanthan), clays (laponite), hydrophilic or amphiphilic crosslinked or non-crosslinked homo- and copolymers of acryloylmethylpropanesulfonic acid (amps) and/or of acrylamide and/or of acrylic acid and/or of salts or esters of acrylic acid (sold under the names Aristoflex AVC, Aristoflex AVS, Aristoflex HMB, Simulgel NS, Simulgel EG, Simulgel 600, Simulgel 800, Pemulen, carbopol, Sepiplus 400, Seppimax zen, Sepiplus S, Cosmedia SP).

Said gelling and/or thickening agent will be present in the composition in a content of about from 0.1% to 10% by total weight of the composition;
one or more surfactant(s), in particular
   anionic surfactants such as isethionates, taurates, sarcosinates, glycinates, glutamates, phosphates (C20-22 alkyl phosphate sold under the name Sensanov WR),
   amphoteric surfactants such as betaine derivatives, amphoacetates,
   nonionic surfactants such as polyglycerol derivatives, sugar derivatives (glucoside or xyloside derivatives sold under the name Montanov 68, Montanov 202, Montanov 82, Montanov L, Easynov), lecithins.

Said surfactant will be present in a content of about from 0.1% to 15%, preferably 0.5% to 10%, by weight, relative to the total weight of the composition;
one or more active agent(s) of natural, biotechnological or synthetic origin having a biological activity and having an effectiveness on the skin via biological sites, for example chosen from vitamins such as vitamin C and its derivatives (ascorbyl glucoside, 3-o-ethyl ascorbic acid, ascorbyl tetraisopalmitate), vitamin A and its derivatives, vitamin E and its derivatives, vitamin B3 or niacinamide, panthenol, trace elements, allantoin, adenosine, peptides (Palmitoyl tetrapeptide-7, Palmitoyl tripeptide-1, Palmitoyl pentapeptide-4, Acetyl dipeptide-1 cetyl ester, Acetyl tetrapeptide-5 sold under the name NP Rigin, Matrixyl 3000, Idealift, Eyeseryl), plant extracts (Glycyrrhiza glabra extract, Centella asiatica leaf extract, rye seed extract), yeast extracts, alpha hydroxy acids such as glycolic or lactic acid, tranexamic acid and its derivatives such as cetyl tranexamic ester, etc.

Said active agent will be present in the composition in a content of about from 0.1% to 10% by total weight of the composition;

Other additives customarily used in cosmetics may also be present in the composition according to the invention, in particular preservatives, antioxidants or fragrances well known in the technical field.

A person skilled in the art is able to choose, from all of these possible additives, both the nature and the amount of those that will be added to the composition, so that the latter retains all of its properties.

The invention is illustrated in a nonlimiting manner by the examples below.

### Example 1: Preparation of an Alcoholic Extract of Flowers of White Lily, Lilium candidum L., According to the Invention

An alcoholic extract of flowers of white lily, Lilium candidum L., according to the invention is prepared by a method comprising the following steps:
a) milling of 200 g of flowers of white lily, Lilium candidum L., to powder;
b) extraction of the powder in 2000 g of 96° ethanol with stirring at 50 °C for 2 h;
Screening of the resulting mixture 1 at 50 μm: the filtrate 1 is set aside; Extraction of the spent grains in 2000 g of 96° ethanol with stirring at 50 °C for 2 h;
Screening of the resulting mixture 2 at 50 μm: the spent grains are discarded;
c) addition of the filtrate 2 to the filtrate 1 and settling of the mixture overnight;
d) filtration of the supernatant at 4 μm;
   Determination of the solids on 3×5 g of extract placed at 110 °C for 3 h;
   Decolorizing of the filtrate for 3 h at ambient temperature with stirring after addition of 20% of active charcoal relative to the dry extract;
   Membrane filtration at 4 and 1 μm: removal of the active charcoal;
e) addition of 180 g of 1,3-propanediol and stirring for half an hour; and
evaporation of the ethanol in a rotary evaporator;
Final filtration at 4 μm.

### Example 2: Test for the Properties of the Extract of Lily According to the Invention Against Oxidative Stress

Protocol: Normal human epidermal keratinocytes derived from two young donors were seeded in a 6-well plate and cultured in a KGM2 supplemented medium (Lonza) for 72 h at 37 °C and 5% CO₂. The cells were then incubated, or not incubated (untreated condition), with 0.25% of extract for 24 h. Each condition was performed in duplicate. The total RNAs were extracted using the RNeasy 96 Plate Extraction Kit (Qiagen) according to the supplier's recommendations. The amount and quality of the RNAs were evaluated by Multiskan Go (Thermo Fischer). The complementary DNAs were synthesized and a transcriptome was produced on an Affymetrix GeneChip Human Transcriptome Array 2.0. The bioinformatics analysis of the genes of which the expression is modulated at least by a factor of 2 (FC: fold change, expression level vs untreated condition) was carried out with the Ingenuity Pathway Analysis software (IPA®, Qiagen).

Results: at 0.25%, the extract is capable of modulating on average the expression of 158 genes (of which 59 were inhibited and 99 stimulated) in normal human keratinocytes after 24 h of treatment.

The IPA software was used to evaluate modifications in the biological activity after treatment with the extract. A map representing modulation of the expression of various biological pathways was established (data not provided). The transcriptome map obtained after 24 h of treatment reveals the modulation of many biological functions with 0.25% of extract. The Madonna lily is in particular capable of modulating 64 canonical signalling pathways.

Among the biological pathways significantly activated by the extract of lily at 0.25% is the NRF2 (nuclear factor-erythroid 2-related factor-2)-mediated oxidative stress response pathway. NRF2 is a transcription factor which, once activated, makes it possible to increase the expression of antioxidant enzymes such as GCLM, GPX, GST, NQO1, SOD and TXNR. NRF2 also activates the transcription of non-enzymatic antioxidant proteins such as ferritin composed in particular of the FTL and FTH1 subunits. The stimulation of this biological pathway for global cell detoxification in normal keratinocytes after 24 h of treatment with the extract at 0.25% gives it antioxidant and anti-aging properties, oxidative stress contributing to aging of the skin.

TABLE I
List of genes involved in the biological pathway for “NRF2-mediated
oxidative stress response” of which the expression is significantly
stimulated by the extract of lily at 0.25%.
Activation of the “NRF2-mediated oxidative stress response”
biological pathway by the extract of lily at 0.25%

| | | FC - | FC - | |
|---|---|---|---|---|
| Symbol | Gene name | Donor 1 | Donor 2 | Average |
| FTH1 | Ferritin heavy chain 1 | 2.5 | 2.5 | 2.5 |
| FTL | Ferritin light chain | 2.2 | 3.0 | 2.6 |
| GCLM | Glutamate-cysteine ligase | 3.2 | 3.2 | 3.2 |
| | modifier subunit | | | |
| GPX2 | Glutathione peroxidase 2 | 3.4 | 3.2 | 3.3 |
| MGST1 | Microsomal glutathione | 2.9 | 3.4 | 3.2 |
| | S-transferase 1 | | | |
| NQO1 | NAD(P)H quinone | 8.3 | 6.6 | 7.5 |
| | dehydrogenase 1 | | | |
| SOD2 | Superoxide dismutase 2 | 2.1 | 1.9 | 2.0 |
| TXNRD1 | Thioredoxin reductase 1 | 2.9 | 3.3 | 3.1 |

NRF2: Nuclear factor-erythroid 2-related factor-2,
FC: expression level vs untreated condition

### Example 3: Anti-Aging Properties of the Extract of Lily According to the Invention in Cellular and Tissue Models of Skin Aging

At the cellular level, the dermis can be divided into two subpopulations of fibroblasts: papillary fibroblasts, located under the epidermis at the level of the papillae and which have a younger phenotype, and reticular fibroblasts, deeper and more differentiated, showing an older phenotype. These two subpopulations express specific markers: CCRL1, NTN and PDPN for papillary fibroblasts and CDH2, CNN and MGP for reticular fibroblasts. Aging of the dermis is characterized in particular by the progressive differentiation of papillary fibroblasts into reticular fibroblasts leading to stiffening of the dermis. This aging can be modeled in the laboratory, at the cellular level, using reticular fibroblasts and, at the tissue level, by reconstructing equivalent skins with a dermis synthesized by reticular fibroblasts. These models can thus be used to identify ingredients with anti-aging properties capable of reversing dermal aging.

Cellular model protocol: Normal human dermal reticular fibroblasts derived from three donors were seeded in a 6-well plate and cultured in DMEM medium supplemented with 5% serum for 72 h at 37 °C and 5% CO₂. The cells were then incubated, or not incubated (untreated condition), with 0.25% of extract for 96 h in DMEM medium supplemented with 2.5% serum. The total RNAs were extracted using the RNeasy 96 Plate Extraction Kit (Qiagen) according to the supplier's recommendations. The amount and quality of the RNAs were evaluated by Experion (Biorad). The complementary DNAs were synthesized (iScript kit, Biorad) then the qPCR reactions were carried out with the SYBR Green method (Biorad) according to this program: 5 min at 95 °C then 35 cycles with 20 seconds at 95 °C, 20 seconds at 60 °C and 20 seconds at 72 °C followed by generation of melting curves. Expression analyses were performed with the iQ5 software (Biorad) using the ΔΔCt method with stably expressed reference genes. The expression of biomarkers specific for papillary fibroblasts (CCRL1, NTN and PDPN) and reticular fibroblasts (CDH2, CNN and MGP) was thus evaluated in the samples.

Results: As shown in FIG. 1, at 0.25%, the extract is able to increase the expression of the papillary markers CCRL1, NTN and PDPN and also to reduce the expression of the reticular markers CDH2, CNN and MGP in reticular fibroblasts after 96 h of treatment. The extract of lily therefore has an ability to reverse the aged phenotype of reticular fibroblasts into a younger phenotype, close to that of papillary fibroblasts.

Tissue model protocol: Normal human dermal papillary or reticular fibroblasts derived from three donors were seeded and cultured in DMEM medium supplemented with 5% serum (HyClone) for 5 days at 37 °C and 5% CO₂. The cells were then incubated, or not incubated (control conditions), with 0.25% extract or with 0.25% propanediol solvent (diluent only condition) for 4 days. Dermis equivalents were generated with the treated or control cells and were then incubated again, or not incubated, with 0.25% of lily extract or of propanediol in DMEM medium supplemented with 5% serum and 1% penicillin-streptomycin (Invitrogen) at 37 °C and 5% CO₂, for 7 days. Normal human epidermal keratinocytes were then seeded onto the dermis equivalents and the cultures were maintained in DMEM/Ham's F12 (3:1) medium supplemented with 5% serum, 1.1 μM hydrocortisone, 1 μM isoproterenol, 0.1 μM insulin (Sigma-Aldrich) and 1% penicillin-streptomycin (Invitrogen) for 2 days. The skins reconstructed with papillary fibroblasts (Pap) or reticular fibroblasts (Ret) were placed at the air/liquid interface and again treated, or not treated, according to the conditions described above, for 14 days before being fixed, dehydrated and then embedded in paraffin. Histological sections were then cut and used for hematoxylin eosin saffron (HES) staining or Ki67 immunolabeling.

The hematoxylin eosin saffron (HES) staining was carried out in order to observe their overall morphology and in particular the thickness of the epidermis which becomes thinner in the models of aged reticular skin (vs. “young” papillary skin) 6. Immunolabeling of Ki67, a cell proliferation marker, was also carried out in order to count the positive cells in the skins reconstructed with papillary fibroblasts. Antigenic unmasking was carried out in citrate buffer at pH 6, before incubation in a blocking solution preventing non-specific binding of antibodies. The sections are then incubated overnight at 4 °C with a primary antibody specifically targeting Ki67 (MIB-1, Dako) and then with the avidin-biotin-peroxidase system, according to the recommendations of the supplier (GE Healthcare). The labeling was visualized by adding AEC (3-amino-9-ethylcarbazole) and the sections were counterstained with hematoxylin.

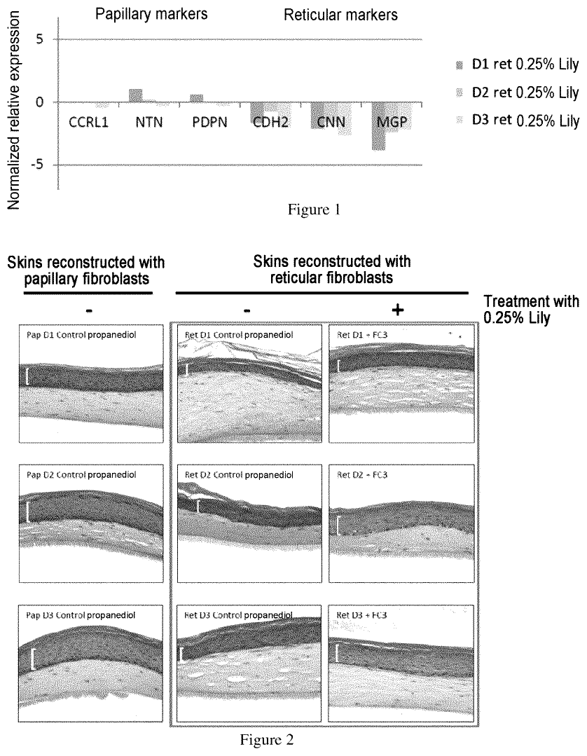

Results: As shown in FIG. 2, at 0.25%, the extract of lily according to the invention is capable of improving the morphology of the epidermis, in particular by promoting thickening thereof (on the right), in models of aged skin reconstructed with reticular fibroblasts compared to untreated conditions (in the center). The skins treated with the extract thus regain a younger morphology, close to those obtained with skins reconstructed with untreated papillary fibroblasts (on the left).

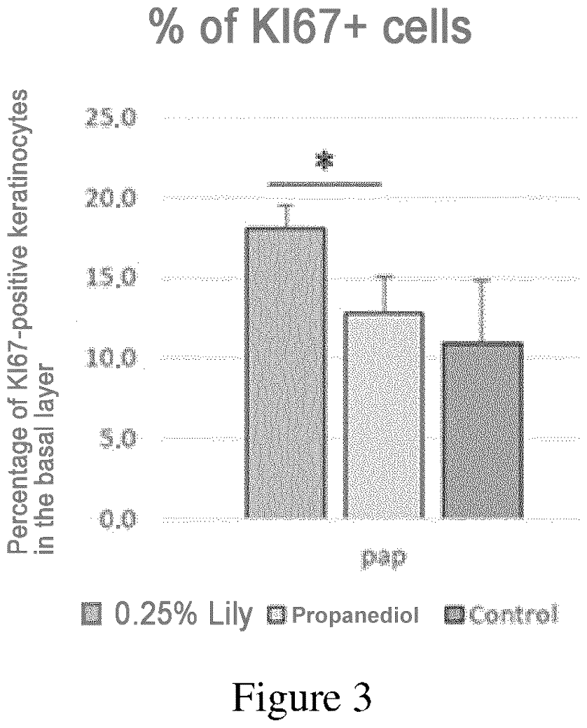

As shown in FIG. 3, at 0.25%, the extract is also capable of increasing the number of cells expressing the Ki67 proliferation marker in the basal layer of skins reconstructed with papillary fibroblasts. It therefore promotes cell renewal.

Thus, at 0.25%, the extract of lily is capable of slowing down dermal aging by modifying a reticular phenotype to a younger papillary phenotype in cellular and tissue models. It therefore has strong anti-aging properties.

### Example 4: Cosmetic Compositions

The following compositions can be prepared in a manner that is conventional for a person skilled in the art. The amounts indicated below are expressed as percentages by weight. The ingredients in capital letters are identified according to the INCI name.

### A—Oil/Water Gel Emulsion

| INCI name | (% W/W) |
|---|---|
| Limnanthes alba (meadowfoam) seed oil | 1-10 |
| Butyrospermum parkii butter (LIPEX SHEASOFT) | 1-10 |
| Butyrospernum parkii butter extract (LIPEX SHEA TRIS) | 1-10 |
| Camellia oleifera seed oil | 1-10 |
| Caprylic/Capric triglyceride | 1-10 |
| Squalane | 1-10 |
| Ammonium acryloyldimethyltaurate/vp copolymer | 0.1-5 |
| Acrylates/C10-30 alkyl acrylate crosspolymer | 0.1-2 |
| Xanthan gum | 0.01-5 |
| Biosaccharide gum-1 | 0.01-5 |
| Sodium hyaluronate | 0.01-3 |
| Glycerin | 1-30 |
| Polyquaternium-51 | 1-10 |
| Tocopheryl acetate | 0.1-5 |
| Niacinamide | 0.1-5 |
| Sphingomonas ferment extract | 0.01-5 |
| Polianthes tuberosa | 0.01-5 |
| Betaine | 0.1-10 |
| Sodium PCA | 0.5-5 |
| Saccharide isomerate | 0.5-5 |
| Extract of lily flowers according to the invention | 0.001-10 |
| Yeast extract | 0.1-5 |
| Glycols (Caprylyl Glycol and/or Pentylene Glycol and/or | 0.1-10 |
| Butylene Glycol and/or propanediol) | |
| Water | qs 100 |

### B—Oil/Water Cream Emulsion

| INCI name | (% w/w) |
|---|---|
| Jojoba esters | 1-5 |
| Limnanthes alba (meadowfoam) seed oil | 0.1-5 |
| Canola oil | 1-10 |
| Argania spinosa kernel oil | 0.1-10 |
| Moringa oil/hydrogenated moringa oil esters | 1-10 |
| C8-12 acid triglyceride | 1-5 |
| Lauroyl lysine | 1-5 |
| Camellia oleifera seed oil | 1-10 |
| Phytosteryl/octyldodecyl lauroyl glutamate | 1-5 |
| Squalane | 1-10 |
| Ammonium acryloyldimethyltaurate/vp copolymer | 1-5 |
| Cetearyl alcohol & cetearyl glucoside | 1-7 |
| Sodium lauroyl glutamate | 0.1-1 |
| Hydrogenated lecithin | 0.1-5 |
| Chondrus crispus (carrageenan) | 0.1-5 |
| Sclerotium gum | 0.01-2 |
| Centella asiatica leaf extract | 0.1-5 |
| Adenosine | 0.1-0.5 |
| Niacinamide | 0.1-5 |
| Rye seed extract | 0.1-5 |
| Palmitoyl tripeptide-1 & palmitoyl tetrapeptide-7 | 1-5 |
| Plankton extract | 0.1-5 |
| Yeast extract | 1-3 |
| Extract of lily flowers according to the invention | 0.001-10 |
| Glycyrrhiza glabra extract | 0.001-5 |
| Tranexamic cetyl ester | 0.001-5 |
| Ascorbyl glucoside | 0.001-5 |
| Water | qs 100 |

These compositions can be applied to the skin daily, in the morning and/or evening.

1-15. (canceled)

16. An alcoholic extract of flowers of white lily, Lilium candidum L., comprising: at least lilidine glycoside and hopantenic acid.

17. The alcoholic extract of white lily flowers as claimed in claim 16, comprising:
40 to 70% by weight of saccharides, preferably 50 to 60% by weight,
1 to 5% by weight of compounds derived from di-O- and tri-O-glycosylated kaempferol, preferably 2.5 to 3.2% by weight,
8 to 15% by weight of lilidine glycoside, preferably 10 to 13% by weight, and
1 to 5% by weight of hopantenic acid, preferably 2 to 3% by weight,
the percentages being expressed by weight, based on the total weight of the extract.

18. The alcoholic extract of white lily flowers as claimed in claim 16, wherein the saccharides comprise glucose, fructose and/or sucrose, preferably 20 to 25% by weight of fructose, 17 to 21% by weight of glucose and 5 to 11% by weight of sucrose.

19. A method for extracting flowers of white lily, Lilium candidum L., comprising the following steps:
a) milling the flowers of white lily, Lilium candidum L.,
b) at least one, preferably at least two, extractions of said milled flowers in the presence of an alcoholic solvent, each extraction being followed by a filtration step;
c) mixing the liquid filtrates from the extractions of step b) and resting of the resulting mixture for at least 6 h, preferably at least 10 h;
d) filtering the mixture obtained in c);
e) removing the monoalcoholic solvent from the mixture obtained in d) and then final diluting in another alcoholic solvent.

20. The method as claimed in claim 19, wherein the flowers of white lily used in step a) are fresh flowers or dried flowers.

21. The method as claimed in claim 19, wherein, in step a), the flowers of white lily are milled to a fineness of less than 2 cm.

22. The method as claimed in claim 19, wherein the alcoholic solvent of step b) is a monoalcohol comprising from 1 to 4 carbon atoms, preferably ethanol.

23. The method as claimed in claim 19, wherein the extractions of step b) are carried out for a period of between 1 and 6 hours, at a temperature of between 40 °C and 60 °C, preferably between 45 and 55 °C.

24. The method as claimed in claim 19, wherein each extraction of step b) is followed by filtration on a 50 μm screen.

25. The method as claimed in claim 19, wherein the filtration carried out in step d) is carried out on a 4 μm filtration screen or membrane.

26. The method as claimed in claim 19, wherein between steps d) and e), a step of decolorizing the filtrate obtained in d), preferably by adsorption on active charcoal, followed by a step of filtering the decolorized filtrate obtained, preferably to 1 μm, are added.

27. The method as claimed in claim 19, wherein the removal in step e) is carried out by evaporation and then the final dilution is carried out in 1,3-propanediol.

28. An alcoholic extract of flowers of white lily, Lilium candidum L., obtained by the method as claimed in claim 19.

29. A cosmetic method for preventing and/or reducing detrimental skin changes due to aging and/or for activating the NRF2-mediated oxidative stress response and/or for promoting epidermal thickening, said method comprising a step of administering an alcoholic extract of flowers of white lily, Lilium candidum L. as claimed in claim 16 to a subject in need thereof.

30. A cosmetic composition comprising, in a cosmetically acceptable carrier, an alcoholic extract of flowers of white lily, Lilium candidum L., as claimed in claim 16.
